(19) United States
(12) Patent Application Publication
(10) Pub. No.: US 2016/0279098 A1
(43) Pub. Date: Sep. 29, 2016

(54) NOVEL METHODS

(71) Applicant: EUTHYMICS BIOSCIENCE, INC., Cambridge, MA (US)

(72) Inventors: Anthony Alexander MCKINNEY, Cambridge, MA (US); Maurizio FAVA, Newton, MA (US); Frank GENTILE, Boxborough, MA (US)

(73) Assignee: Euthymics Bioscience, Inc., Cambridge, MA (US)

(21) Appl. No.: 15/035,894

(22) PCT Filed: Nov. 11, 2014
(86) PCT No.: PCT/US2014/065095

§ 371 Date: May 11, 2016

Related U.S. Application Data
(60) Provisional application No. 62/077,107, filed on Nov. 7, 2014.
(60) Provisional application No. 61/902,762, filed on Nov. 11, 2013.

(51) Int. Cl. A61K 31/403; A61K 45/06

(52) U.S. Cl. CPC A61K 31/403; A61K 45/06

(57) ABSTRACT

Provided is a method of treating female sexual dysfunction comprising administering a therapeutically effective amount of (+)-1-(3,4-dichlorophenyl)-3-azabicyclo[3.1.0]hexane, in free or pharmaceutically acceptable salt form, substantially free of the corresponding (−) enantiomer, to a female in need thereof.

This application claims priority to U.S. Provisional Application No. 62/077,107, filed Nov. 7, 2014, and U.S. Provisional Application No. 61/902,762, filed Nov. 11, 2013, the contents of each of which are incorporated herein by reference.

### BACKGROUND

Females can suffer from female sexual dysfunction (FSD). It has been reported that approximately 40 million American women are affected by FSD and that sexual dysfunction is more prevalent in women (43%) than in men (31%) and increases as women age.

There is thus a need for methods to treat female sexual dysfunction.

### BRIEF SUMMARY

Provided is a method of treating female sexual dysfunction comprising administering a therapeutically effective amount of (+)-1-(3,4-dichlorophenyl)-3-azabicyclo[3.1.0]hexane, in free or pharmaceutically acceptable salt form, to a female in need thereof.

Further provided is a method of treating female sexual dysfunction comprising administering a therapeutically effective amount of (+)-1-(3,4-dichlorophenyl)-3-azabicyclo[3.1.0]hexane, in free or pharmaceutically acceptable salt form, substantially free of the corresponding (−) enantiomer, to a female in need thereof.

Further areas of applicability of the present disclosure will become apparent from the detailed description provided hereinafter. It should be understood that the detailed description and specific examples, while indicating the preferred embodiment of this disclosure, are intended for purposes of illustration only and are not intended to limit the scope of this disclosure.

### BRIEF DESCRIPTION OF THE DRAWINGS

The present disclosure will become more fully understood from the detailed description and the accompanying drawings, wherein:

FIG. 1 shows SFI Score for Item 1: Amitifadine 100 mg vs. Paroxetine.

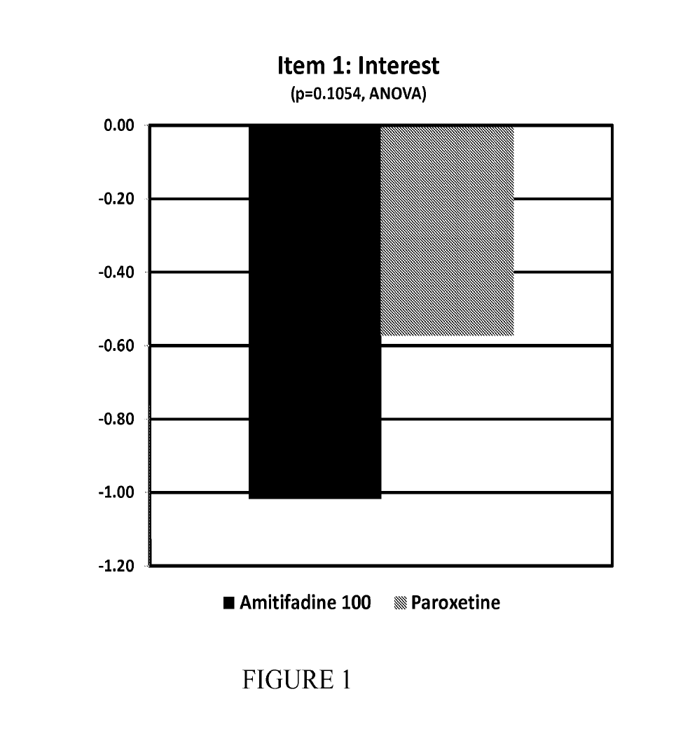

FIG. 2 shows SFI Score for Item 2: Amitifadine 100 mg vs. Paroxetine.

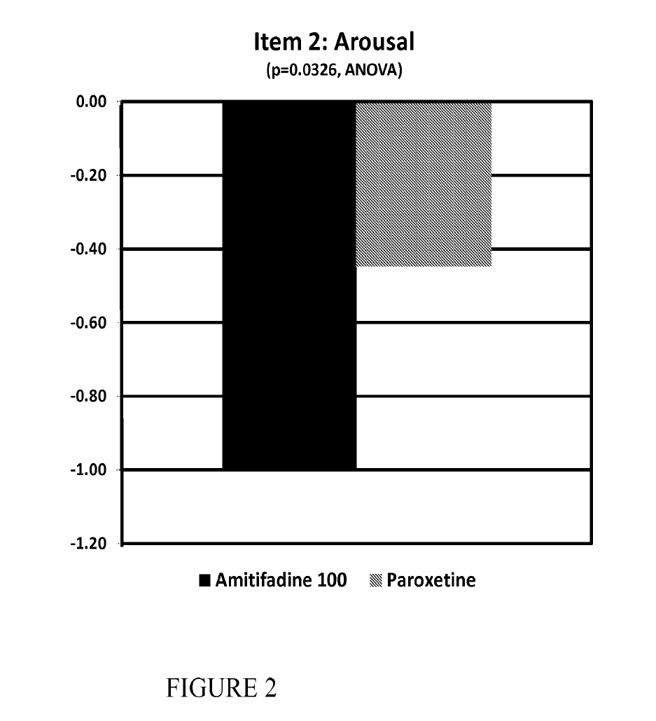

FIG. 3 shows SFI Score for Item 3: Amitifadine 100 mg vs. Paroxetine.

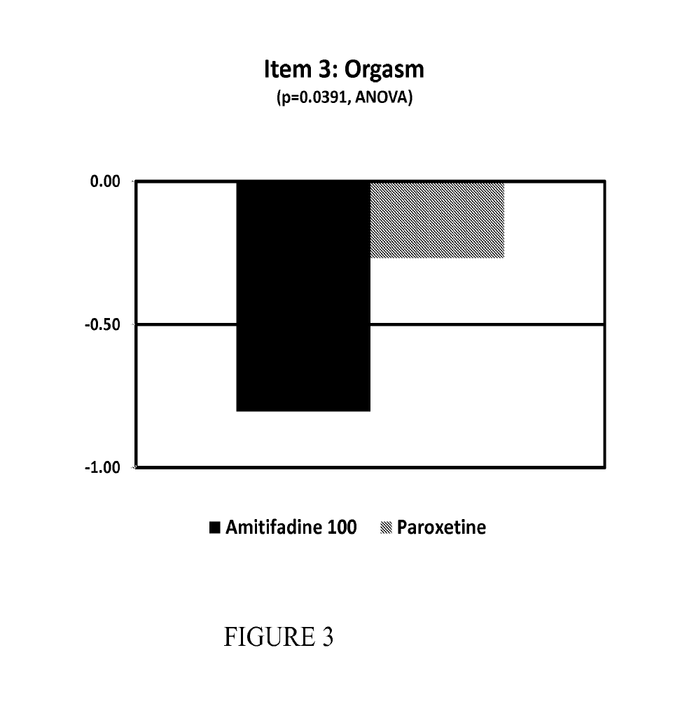

FIG. 4 shows SFI Score for Item 4: Amitifadine 100 mg vs. Paroxetine.

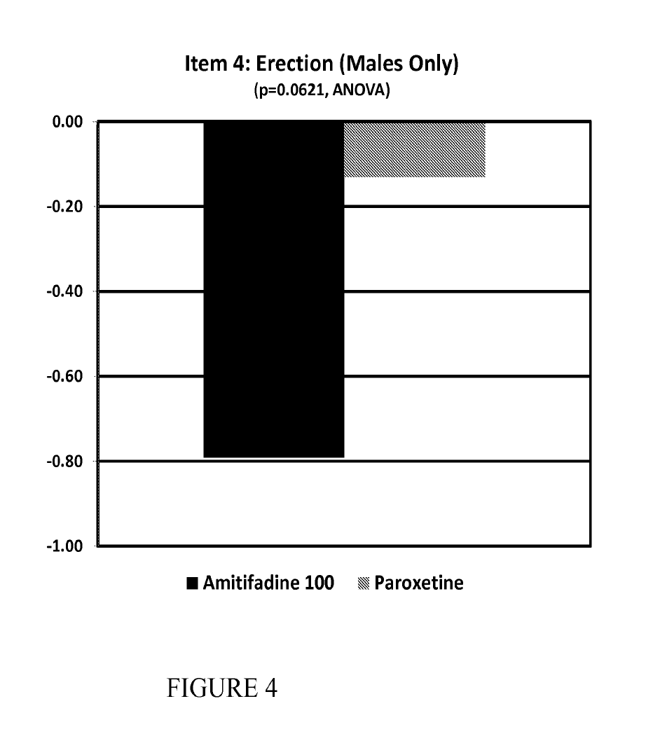

FIG. 5 shows SFI Score for Item 5: Amitifadine 100 mg vs. Paroxetine.

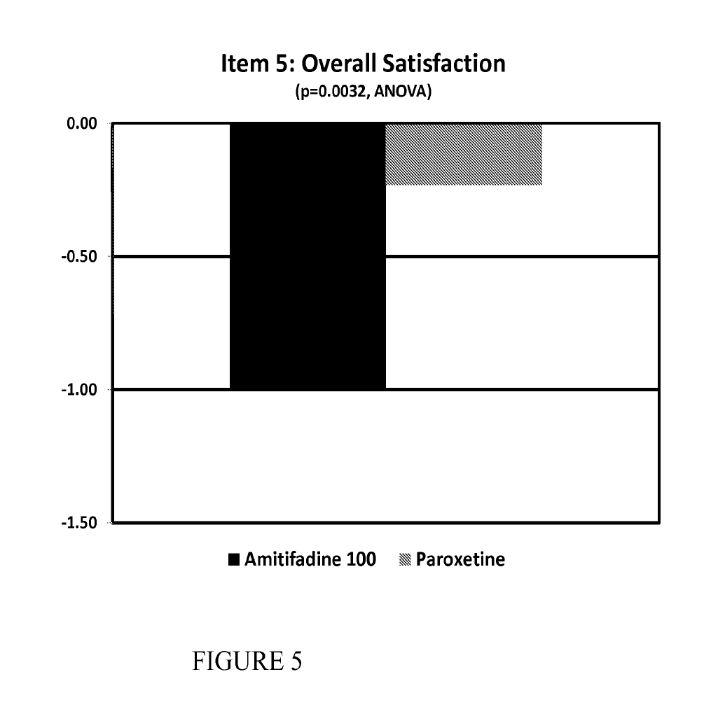

FIG. 6 shows SFI Total Score: Amitifadine 100 mg vs. Paroxetine.

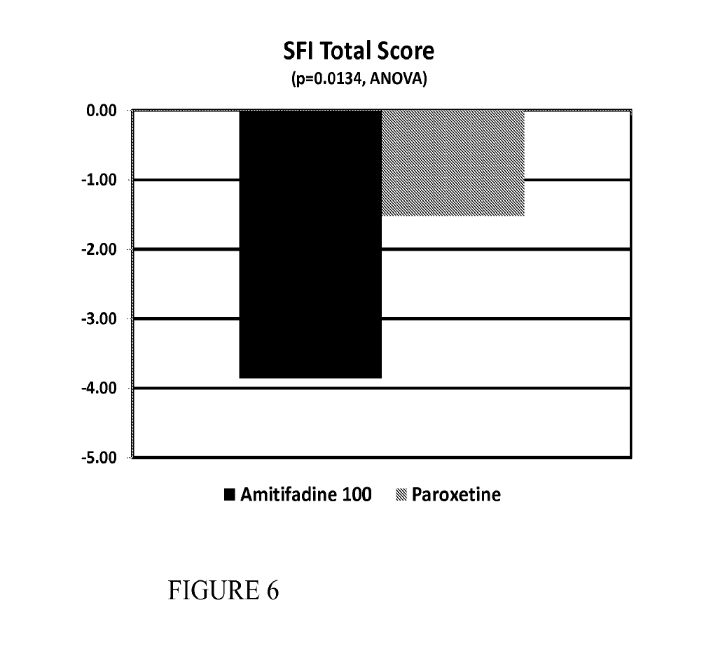

FIG. 7 shows SFI Score for Item 1 by Gender: Amitifadine 100 mg vs. Placebo.

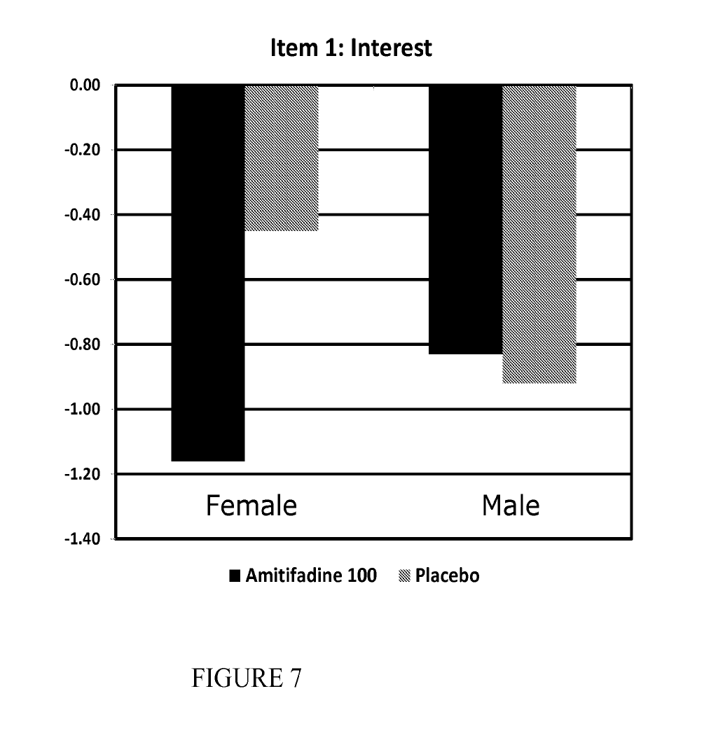

FIG. 8 shows SFI Score for Item 2 by Gender: Amitifadine 100 mg vs. Placebo.

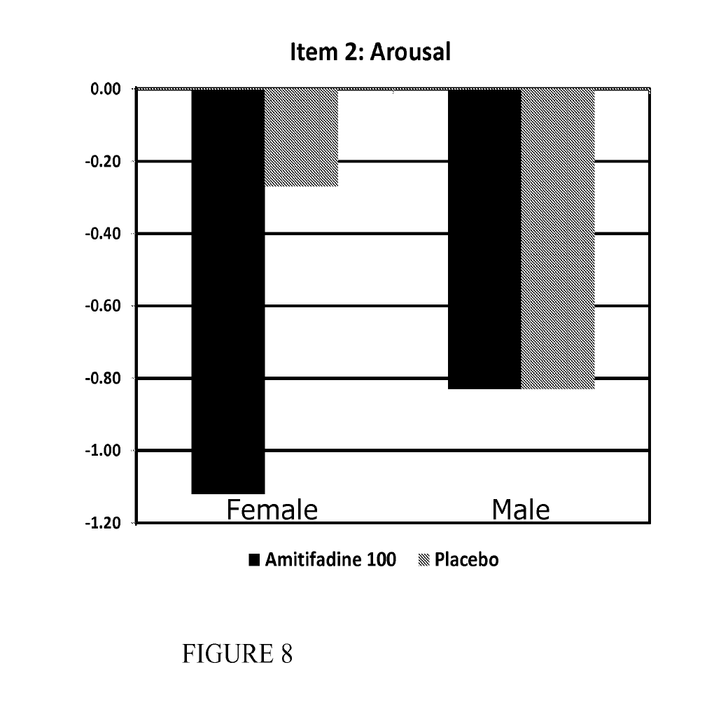

FIG. 9 shows SFI Score for Item 3 by Gender: Amitifadine 100 mg vs. Placebo.

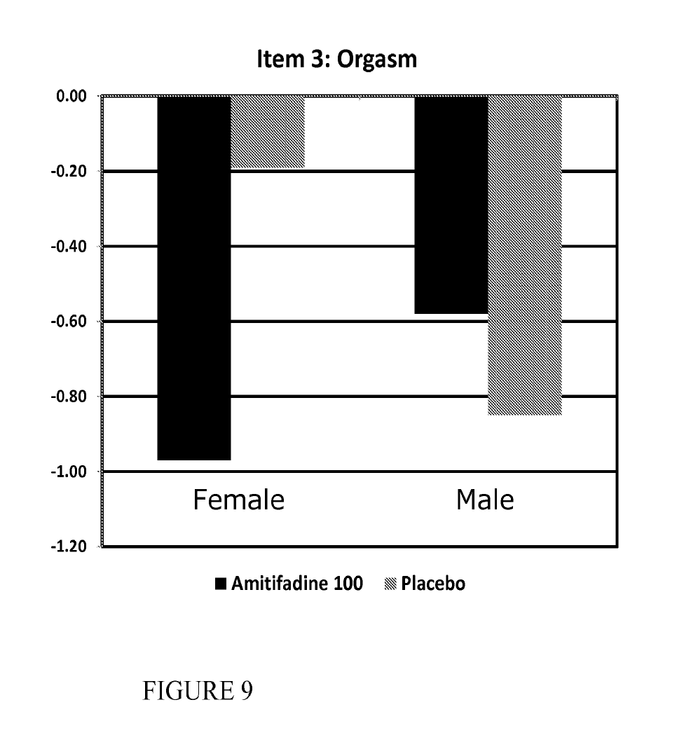

FIG. 10 shows SFI Score for Item 4 by Gender: Amitifadine 100 mg vs. Placebo.

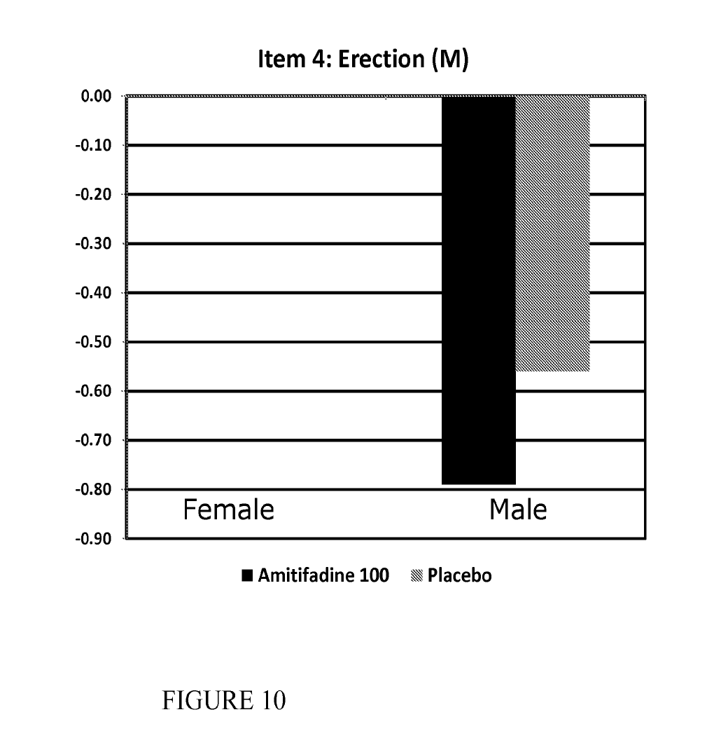

FIG. 11 shows SFI Score for Item 5 by Gender: Amitifadine 100 mg vs. Placebo.

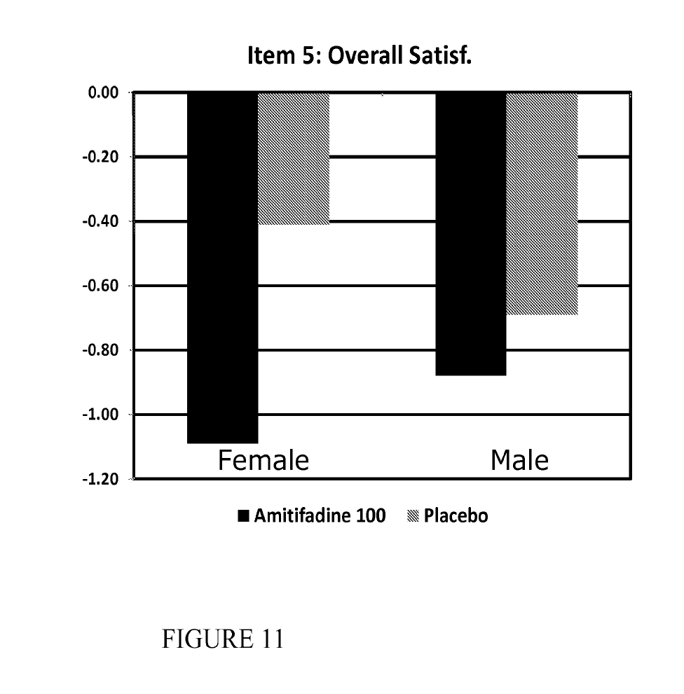

FIG. 12 shows SFI Total Score by Gender: Amitifadine 100 mg vs. Placebo

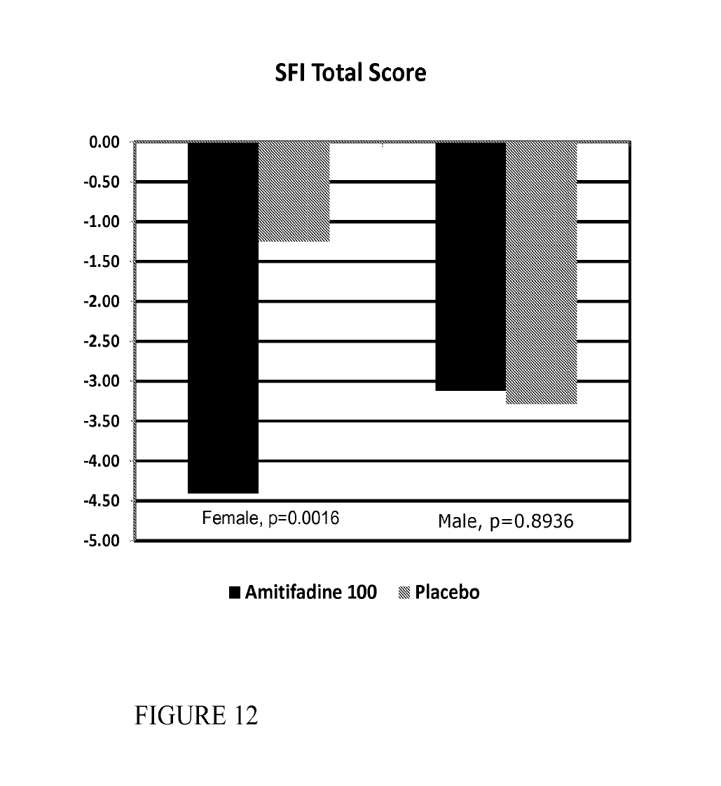

### DETAILED DESCRIPTION

The following description of the preferred embodiment(s) is merely exemplary in nature and is in no way intended to limit this disclosure, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

There are both excitatory and inhibitory factors that may influence sexual desire. Excitatory factors include testosterone, melanocortin, oxytocin, dopamine, and norepinephrine. Inhibitory factors include serotonin (5-hydroxytryptamine (5-HT)), prolactin, and endogenous opioids.

It has been reported that in both in vitro and in vivo studies dopamine inhibits prolactin (PRL) release. Dopamine binds to the D2 receptor subclass expressed on lactotroph cell membranes. Activation of that receptor results in suppression of PRL gene expression and inhibition of PRL exocytosis.

Serotonin has a stimulatory role in prolactin secretion. Changes in sexual desire, sexual performance, and sexual satisfaction may occur as a side effect of treatment with a selective serotonin reuptake inhibitor (SSRI).

(+)-1-(3,4-dichlorophenyl)-3-azabicyclo[3.1.0]hexane, also known as (1R,5S)-1-(3,4-dichlorophenyl)-3-azabicyclo[3.1.0]hexane, also known amitifadine, is shown as Formula I below.

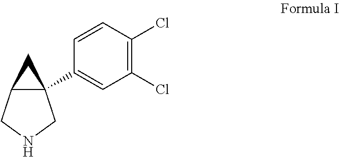 [SMILES: Clc1ccc([C@]23CNC[C@H]2C3)cc1Cl]

“(+)-1-(3,4-dichlorophenyl)-3-azabicyclo[3.1.0]hexane,” “(1R,5S)-1-(3,4-dichlorophenyl)-3-azabicyclo[3.1.0]hexane,” and “amitifadine” are used interchangeably herein. (+)-1-(3,4-dichlorophenyl)-3-azabicyclo[3.1.0]hexane is an unbalanced triple reuptake inhibitor with the greatest potency towards serotonin reuptake (5-HT), half as much towards norepinephrine reuptake (NE), and one eighth towards dopamine reuptake (DA). (+)-1-(3,4-dichlorophenyl)-3-azabicyclo[3.1.0]hexane hydrochloride is reported to inhibit the reuptake of [³H]serotonin, [³H]norepinephrine, and [³H]dopamine in human embryonic kidney (HEK) 293 cells expressing the corresponding human recombinant transporters at IC₅₀ values of 12, 23, and 96 nm, respectively.

In a recent clinical trial for use of amitifadine in the treatment of major depressive disorder, individuals receiving amitifadine 100 mg had significantly less overall worsening of sexual function than those receiving paroxetine as assessed with the Sexual Functioning Inventory (SFI). In addition, women experienced significant improvement in SFI on 100 mg amitifadine from baseline compared with placebo, which is unexpected considering that amitifadine more strongly inhibits serotonin reuptake than dopamine reuptake.

Without being bound by theory, amitifadine, by inhibiting dopamine reuptake, may enhance the synaptic availability of dopamine enough to inhibit prolactin (PRL) release. As prolactin is an inhibitory factor on sexual desire, inhibiting its release may enhance sexual desire.

(+)-1-(3,4-dichlorophenyl)-3-azabicyclo[3.1.0]hexane may be synthesized as described in U.S. Patent Publication No. 2008/0045725, incorporated herein by reference in its entirety.

As used herein, “(+)-1-(3,4-dichlorophenyl)-3-azabicyclo[3.1.0]hexane, in free or pharmaceutically acceptable salt form, substantially free of the corresponding (−) enantiomer” means containing no more than 5% w/w (weight/weight) of the corresponding (−) enantiomer, in free or pharmaceutically acceptable salt form, e.g., no more than 2% w/w of the corresponding (−) enantiomer, in free or pharmaceutically acceptable salt form, e.g., no more than 1% w/w of the corresponding (−) enantiomer, in free or pharmaceutically acceptable salt form.

As used herein, “(+)-1-(3,4-dichlorophenyl)-3-azabicyclo[3.1.0]hexane” embraces the compound in any form, for example, free or pharmaceutically acceptable salt form, e.g., as a pharmaceutically acceptable acid addition salt. Pharmaceutically acceptable salts are known in the art and include salts that are physiologically acceptable at the dosage amount and form to be administered, for example, hydrochloride salts.

As used herein, “(+)-1-(3,4-dichlorophenyl)-3-azabicyclo[3.1.0]hexane” is also to be understood as embracing the compound in crystalline and amorphous form including, for example, polymorphs, solvates (including hydrates), unsolvated polymorphs (including anhydrates), conformational polymorphs, and amorphous forms of the compounds, as well as mixtures thereof “Crystalline form” and “polymorph” may be used interchangeably herein, and are meant to include all crystalline forms of (+)-1-(3,4-dichlorophenyl)-3-azabicyclo[3.1.0]hexane, in free or pharmaceutically acceptable salt form, including, for example, polymorphs, solvates (including hydrates), unsolvated polymorphs (including anhydrates), and conformational polymorphs, as well as mixtures thereof, unless a particular crystalline form is referred to.

(+)-1-(3,4-dichlorophenyl)-3-azabicyclo[3.1.0]hexane exists in at least three polymorphic forms, labeled polymorphs A, B, and C, as disclosed in U.S. Patent Publication Nos. 2007/0043100 and 2009/0326245, incorporated herein by reference in their entirety.

Crystalline and amorphous forms of (+)-1-(3,4-dichlorophenyl)-3-azabicyclo[3.1.0]hexane may be used in any combination or in forms that are substantially free of one or more of the other crystalline forms or free of the amorphous form.

As used herein, “substantially free of other polymorphic forms” means that the crystalline material contains no more than 5% w/w of any other crystalline form, e.g., no more than 2% w/w of any other crystalline form, e.g., no more than 1% w/w of any other crystalline form.

(+)-1-(3,4-dichlorophenyl)-3-azabicyclo[3.1.0]hexane may in some cases also exist in prodrug form. Prodrugs are considered to be any covalently bonded carriers that release the active parent drug in vivo.

As used herein, “therapeutically effective amount” refers to an amount effective, when administered to a human or non-human patient, to provide a therapeutic benefit such as amelioration of symptoms. The specific dose of substance administered to obtain a therapeutic benefit will, of course, be determined by the particular circumstances surrounding the case, including, for example, the specific substance administered, the route of administration, the condition being treated, and the individual being treated.

As used herein, “concurrently” means the compounds are administered simultaneously or within the same composition. In some embodiments, the compounds are administered simultaneously. In some embodiments, the compounds are administered within the same composition.

As used herein, “female sexual dysfunction” generally refers to impairment of sexual function. For example, “female sexual dysfunction” includes, but is not limited to, hypoactive sexual desire, sexual aversion disorder, sexual arousal disorder, orgasmic disorder, sexual pain disorder (including, e.g., dyspareunia, vaginismus, and noncoital sexual pain disorder), and combinations thereof.

As used herein, “female” includes premenopausal and post-menopausal females.

As used herein, “treatment” and “treating” embrace enhancing or improving sexual response and/or sexual pleasure and/or sensation. “Enhancing” or “improving” sexual response and/or sexual pleasure and/or sensation means that administration of (+)-1-(3,4-dichlorophenyl)-3-azabicyclo[3.1.0]hexane increases the female's satisfaction with the sexual activity as compared to the activity when in the absence of (+)-1-(3,4-dichlorophenyl)-3-azabicyclo[3.1.0]hexane. This includes, e.g., enhancement of vaginal wetness, warmth, engorgement, sensitivity, sensation, tingling, arousal, orgasm, quicker to arousal, quicker to orgasm, and enhancement of any of the above-mentioned conditions (for example, clitoral and labial sensation or vaginal smooth muscle relaxation), etc.

Provided herein is a method (Method 1) of prophyalaxis or treatment of female sexual dysfunction comprising administering a therapeutically effective amount of (+)-1-(3,4-dichlorophenyl)-3-azabicyclo[3.1.0]hexane, in free or pharmaceutically acceptable salt form, to a female in need thereof.

Further provided is Method 1 as follows:
1.1 Method 1 wherein the method is for the treatment of female sexual dysfunction.
1.2 Method 1 or 1.1 wherein the therapeutically effective amount of (+)-1-(3,4-dichlorophenyl)-3-azabicyclo[3.1.0]hexane, in free or pharmaceutically acceptable salt form, comprises less than or equal to 5% w/w of the corresponding (−) enantiomer.
1.3 Method 1, 1.1, or 1.2 wherein the therapeutically effective amount of (+)-1-(3,4-dichlorophenyl)-3-azabicyclo[3.1.0]hexane, in free or pharmaceutically acceptable salt form, comprises less than or equal to 2% w/w of the corresponding (−) enantiomer.
1.4 Method 1 or 1.1-1.3 wherein the therapeutically effective amount of (+)-1-(3,4-dichlorophenyl)-3-azabicyclo[3.1.0]hexane, in free or pharmaceutically acceptable salt form, comprises less than or equal to 1% w/w of the corresponding (−) enantiomer.
1.5 Method 1 or 1.1-1.4 wherein (+)-1-(3,4-dichlorophenyl)-3-azabicyclo[3.1.0]hexane is in pharmaceutically acceptable salt form.
1.6 Method 1 or 1.1-1.5 wherein (+)-1-(3,4-dichlorophenyl)-3-azabicyclo[3.1.0]hexane in pharmaceutically acceptable salt form is an acid addition salt.
1.7 Method 1 or 1.1-1.6 wherein (+)-1-(3,4-dichlorophenyl)-3-azabicyclo[3.1.0]hexane in pharmaceutically acceptable salt form is (+)-1-(3,4-dichlorophenyl)-3-azabicyclo[3.1.0]hexane hydrochloride.
1.8 Method 1 or 1.1-1.7 wherein (+)-1-(3,4-dichlorophenyl)-3-azabicyclo[3.1.0]hexane in pharmaceutically acceptable salt form is crystalline.
1.9 Method 1 or 1.1-1.8 wherein (+)-1-(3,4-dichlorophenyl)-3-azabicyclo[3.1.0]hexane, in free or pharmaceutically acceptable salt form, is Polymorph A.
1.10 Method 1 or 1.1-1.8 wherein (+)-1-(3,4-dichlorophenyl)-3-azabicyclo[3.1.0]hexane, in free or pharmaceutically acceptable salt form, is Polymorph B.
1.11 Method 1 or 1.1-1.8 wherein (+)-1-(3,4-dichlorophenyl)-3-azabicyclo[3.1.0]hexane, in free or pharmaceutically acceptable salt form, is Polymorph C.
1.12 Method 1 or 1.1-1.11 comprising administering 10 mg to 1800 mg, e.g., 25 mg to 1800 mg, e.g., 10 mg to 1600 mg, e.g., 10 mg to 1200 mg, e.g., 50 mg to 1200 mg, e.g., 75 mg to 1000 mg, e.g., 75 mg to 800 mg, e.g., 75 mg to 500 mg, e.g., 100 mg to 500 mg, e.g., 100 mg to 400 mg, e.g., 100 mg to 300 mg, e.g., 100 mg to 200 mg, e.g., 10 mg to 100 mg, 200 mg, or 300 mg, e.g., 25 mg to 100 mg, 200 mg, or 300 mg, e.g., 50 mg to 100 mg, 200 mg, or 300 mg, of (+)-1-(3,4-dichlorophenyl)-3-azabicyclo[3.1.0]hexane, in free or pharmaceutically acceptable salt form.
1.13 Method 1 or 1.1-1.12 comprising administering 75 mg to 1000 mg, e.g., 100 mg to 600 mg, e.g., 100 mg to 400 mg, e.g., 100 mg to 200 mg, of (+)-1-(3,4-dichlorophenyl)-3-azabicyclo[3.1.0]hexane, in free or pharmaceutically acceptable salt form.
1.14 Method 1 or 1.1-1.13 comprising administering 25 mg to 600 mg, 50 mg to 600 mg, e.g., 25 mg to 200 mg, e.g., 50 mg to 200 mg, e.g., 50 mg to 100 mg, e.g., 100 mg to 600 mg, e.g., 100 mg to 400 mg, e.g., 100 mg to 200 mg, of (+)-1-(3,4-dichlorophenyl)-3-azabicyclo[3.1.0]hexane, in free or pharmaceutically acceptable salt form.
1.15 Method 1 or 1.1-1.14 comprising administering 100 mg to 600 mg, e.g., 100 mg to 400 mg, e.g., 100 mg to 300 mg, e.g., 100 mg to 200 mg, e.g., 100 mg, 200 mg, 300 mg, or 400 mg, of (+)-1-(3,4-dichlorophenyl)-3-azabicyclo[3.1.0]hexane, in free or pharmaceutically acceptable salt form.
1.16 Method 1 or 1.1-1.15 comprising administering 100 mg to 200 mg, e.g., 100 mg, of (+)-1-(3,4-dichlorophenyl)-3-azabicyclo[3.1.0]hexane, in free or pharmaceutically acceptable salt form.
1.17 Method 1 or 1.1-1.16 comprising administering (+)-1-(3,4-dichlorophenyl)-3-azabicyclo[3.1.0]hexane, in free or pharmaceutically acceptable salt form, once, twice, three, or four times daily.
1.18 Method 1 or 1.1-1.17 comprising administering (+)-1-(3,4-dichlorophenyl)-3-azabicyclo[3.1.0]hexane, in free or pharmaceutically acceptable salt form, at least once daily.
1.19 Method 1 or 1.1-1.18 comprising administering (+)-1-(3,4-dichlorophenyl)-3-azabicyclo[3.1.0]hexane, in free or pharmaceutically acceptable salt form, at least twice daily.
1.20 Method 1 or 1.1-1.19 comprising administering (+)-1-(3,4-dichlorophenyl)-3-azabicyclo[3.1.0]hexane, in free or pharmaceutically acceptable salt form, at least three times daily.
1.21 Method 1 or 1.1-1.20 comprising administering (+)-1-(3,4-dichlorophenyl)-3-azabicyclo[3.1.0]hexane, in free or pharmaceutically acceptable salt form, four times daily.
1.22 Method 1 or 1.1-1.21 comprising administering (+)-1-(3,4-dichlorophenyl)-3-azabicyclo[3.1.0]hexane, in free or pharmaceutically acceptable salt form, 24 hours or less, e.g., 12 hours, e.g., 10 hours, e.g., 8 hours, e.g., 6 hours, e.g., 4 hours, e.g., 3 hours, e.g., 2 hours, e.g., 1 hour or less, before sexual activity.
1.23 Method 1 or 1.1-1.22 wherein (+)-1-(3,4-dichlorophenyl)-3-azabicyclo[3.1.0]hexane, in free or pharmaceutically acceptable salt form, is administered concurrently or sequentially, in either order, with another treatment for female sexual dysfunction.
1.24 Method 1 or 1.1-1.23 wherein (+)-1-(3,4-dichlorophenyl)-3-azabicyclo[3.1.0]hexane, in free or pharmaceutically acceptable salt form, is administered concurrently or sequentially, in either order, with a hormone or a derivative thereof (e.g., estrogen, testosterone (e.g., micronized oral testosterone), methyltestosterone, testosterone propionate, dehydroepiandrosterone, dehydroepiandrosterone sulfate), a prostaglandin (e.g., alprostadil), a melanocortin agonist (e.g., bremelanotide), a phosphodiesterase inhibitor (e.g., a PDE5 inhibitor, e.g., Viagra, or, e.g., a PDE1 inhibitor), a dopamine-norepinephrine reuptake inhibitor (e.g., bupropion), a 5-hydroxytryptamine₁ₐ receptor agonist, a dopamine agonist (e.g., apomorphine), a serotonin antagonist and reuptake inhibitor (e.g., trazodone), a lubricant, vitamin E, mineral oil, or a combination thereof
1.25 Method 1 or 1.1-1.24 wherein the female has major depressive disorder.
1.26 Method 1 or 1.1-1.25 wherein the female is taking an anti-hypertensive (e.g., a diuretic, a beta blocker, a calcium-channel blocker, an anti-adrenergic), an anticholinergic (e.g., propantheline, methantheline), a barbiturate, a benzodiazepine (e.g, diazepam, alprazolam), an anti-depressant (e.g., a selective serotonin re-uptake inhibitor, a serotonin-norepinephrine re-uptake inhibitor, a tricyclic antidepressant, a tetracyclic antidepressant, or a monoamine oxidase inhibitor, e.g., a selective serotonin re-uptake inhibitor, e.g., paroxetine), a chemotherapeutic (e.g., cyclophosphamide, an anti-estrogen), or an opiate (e.g., morphine, codeine, methadone), e.g., wherein the female is taking an anti-depressant (e.g., a selective serotonin re-uptake inhibitor, a serotonin-norepinephrine re-uptake inhibitor, a tricyclic antidepressant, a tetracyclic antidepressant, or a monoamine oxidase inhibitor, e.g., a selective serotonin re-uptake inhibitor, e.g., paroxetine).
1.27 Method 1 or 1.1-1.26 wherein the sexual dysfunction is a side effect of a drug, e.g., an anti-hypertensive (e.g., a diuretic, a beta blocker, a calcium-channel blocker, an anti-adrenergic), an anticholinergic (e.g., propantheline, methantheline), a barbiturate, a benzodiazepine (e.g, diazepam, alprazolam), an anti-depressant (e.g., a selective serotonin re-uptake inhibitor, a serotonin-norepinephrine re-uptake inhibitor, a tricyclic antidepressant, a tetracyclic antidepressant, or a monoamine oxidase inhibitor, e.g., selective serotonin re-uptake inhibitor, e.g., paroxetine), a chemotherapeutic (e.g., cyclophosphamide, an anti-estrogen), or an opiate (e.g., morphine, codeine, methadone), e.g., wherein the sexual dysfunction is a side effect of an anti-depressant (e.g., a selective serotonin re-uptake inhibitor, a serotonin-norepinephrine re-uptake inhibitor, a tricyclic antidepressant, a tetracyclic antidepressant, or a monoamine oxidase inhibitor, e.g., a selective serotonin re-uptake inhibitor, e.g., paroxetine).
1.28 Method 1.26 or 1.27 comprising administering a subthreshold amount of (+)-1-(3,4-dichlorophenyl)-3-azabicyclo[3.1.0]hexane, in free or pharmaceutically acceptable salt form.
1.29 Method 1.28 wherein the subthreshold amount is the subthreshold amount of (+)-1-(3,4-dichlorophenyl)-3-azabicyclo[3.1.0]hexane, in free or pharmaceutically acceptable salt form, for treatment of depression.
1.30 Method 1.28 wherein the subthreshold amount is the subthreshold amount of (+)-1-(3,4-dichlorophenyl)-3-azabicyclo[3.1.0]hexane, in free or pharmaceutically acceptable salt form, for treatment of female sexual dysfunction.
1.31 Any of Method 1.26-1.30 comprising administering 10 mg to 400 mg, e.g., 25 mg to 400 mg, e.g., 25 mg to 300 mg, e.g., 50 mg to 400 mg, e.g., 50 mg to 300 mg, e.g., 50 mg to 200 mg, e.g., 50 to 100 mg, e.g., 100 mg to 400 mg, e.g., 100 mg to 300 mg, e.g., 100 mg to 200 mg, e.g., 100 mg, 200 mg, 300 mg, or 400 mg, of (+)-1-(3,4-dichlorophenyl)-3-azabicyclo[3.1.0]hexane, in free or pharmaceutically acceptable salt form.

Also provided herein is use of (+)-1-(3,4-dichlorophenyl)-3-azabicyclo[3.1.0]hexane, in free or pharmaceutically acceptable salt form, for treatment or prophylaxis of female sexual dysfunction, e.g., for use in any of Method 1 or 1.1-1.31.

Also provided herein is use of (+)-1-(3,4-dichlorophenyl)-3-azabicyclo[3.1.0]hexane, in free or pharmaceutically acceptable salt form, in the manufacture of a medicament for treatment or prophylaxis of female sexual dysfunction, e.g., for use in any of Method 1 or 1.1-1.31.

Also provided is a pharmaceutical composition comprising (+)-1-(3,4-dichlorophenyl)-3-azabicyclo[3.1.0]hexane, in free or pharmaceutically acceptable salt form, in combination with a pharmaceutically acceptable diluent or carrier for use in treatment or prophylaxis of female sexual dysfunction, e.g., for use in any of Method 1 or 1.1-1.31.

Also provided herein is use of (+)-1-(3,4-dichlorophenyl)-3-azabicyclo[3.1.0]hexane, in free or pharmaceutically acceptable salt form, for treating female sexual dysfunction, e.g., for use in any of Method 1 or 1.1-1.31.

Also provided herein is use of (+)-1-(3,4-dichlorophenyl)-3-azabicyclo[3.1.0]hexane, in free or pharmaceutically acceptable salt form, in the manufacture of a medicament for treating female sexual dysfunction, e.g., for use in any of Method 1 or 1.1-1.31.

Also provided is a pharmaceutical composition comprising (+)-1-(3,4-dichlorophenyl)-3-azabicyclo[3.1.0]hexane, in free or pharmaceutically acceptable salt form, in combination with a pharmaceutically acceptable diluent or carrier for use in treating female sexual dysfunction, e.g., for use in any of Method 1 or 1.1-1.31.

Provided herein is a method (Method 2) of prophyalaxis or treatment of female sexual dysfunction comprising administering a therapeutically effective amount of (+)-1-(3,4-dichlorophenyl)-3-azabicyclo[3.1.0]hexane, in free or pharmaceutically acceptable salt form, substantially free of the corresponding (−) enantiomer, to a female in need thereof.

Further provided is Method 2 as follows:
2.1 Method 2 wherein the method is for the treatment of female sexual dysfunction.
2.2 Method 2 or 2.1 wherein the therapeutically effective amount of (+)-1-(3,4-dichlorophenyl)-3-azabicyclo[3.1.0]hexane, in free or pharmaceutically acceptable salt form, comprises less than or equal to 2% w/w of the corresponding (−) enantiomer.
2.3 Method 2, 2.1, or 2.2 wherein the therapeutically effective amount of (+)-1-(3,4-dichlorophenyl)-3-azabicyclo[3.1.0]hexane, in free or pharmaceutically acceptable salt form, comprises less than or equal to 1% w/w of the corresponding (−) enantiomer.
2.4 Method 2 or 2.1-2.3 wherein (+)-1-(3,4-dichlorophenyl)-3-azabicyclo[3.1.0]hexane is in pharmaceutically acceptable salt form substantially free of the corresponding (−) enantiomer.
2.5 Method 2 or 2.1-2.4 wherein (+)-1-(3,4-dichlorophenyl)-3-azabicyclo[3.1.0]hexane in pharmaceutically acceptable salt form is an acid addition salt substantially free of the corresponding (−) enantiomer.
2.6 Method 2 or 2.1-2.5 wherein (+)-1-(3,4-dichlorophenyl)-3-azabicyclo[3.1.0]hexane in pharmaceutically acceptable salt form is (+)-1-(3,4-dichlorophenyl)-3-azabicyclo[3.1.0]hexane hydrochloride substantially free of the corresponding (−) enantiomer.
2.7 Method 2 or 2.1-2.6 wherein (+)-1-(3,4-dichlorophenyl)-3-azabicyclo[3.1.0]hexane in pharmaceutically acceptable salt form is crystalline substantially free of the corresponding (−) enantiomer.
2.8 Method 2 or 2.1-2.7 wherein (+)-1-(3,4-dichlorophenyl)-3-azabicyclo[3.1.0]hexane, in free or pharmaceutically acceptable salt form, is Polymorph A substantially free of other polymorphic forms and substantially free of the corresponding (−) enantiomer.
2.9 Method 2 or 2.1-2.7 wherein (+)-1-(3,4-dichlorophenyl)-3-azabicyclo[3.1.0]hexane, in free or pharmaceutically acceptable salt form, is Polymorph B substantially free of other polymorphic forms and substantially free of the corresponding (−) enantiomer.
2.10 Method 2 or 2.1-2.7 wherein (+)-1-(3,4-dichlorophenyl)-3-azabicyclo[3.1.0]hexane, in free or pharmaceutically acceptable salt form, is Polymorph C substantially free of other polymorphic forms and substantially free of the corresponding (−) enantiomer.
2.11 Method 2 or 2.1-2.10 comprising administering 10 mg to 1800 mg, e.g., 25 mg to 1800 mg, e.g., 10 mg to 1600 mg, e.g., 10 mg to 1200 mg, e.g., 50 mg to 1200 mg, e.g., 75 mg to 1000 mg, e.g., 75 mg to 800 mg, e.g., 75 mg to 500 mg, e.g., 100 mg to 500 mg, e.g., 100 mg to 400 mg, e.g., 100 mg to 300 mg, e.g., 100 mg to 200 mg, e.g., 10 mg to 100 mg, 200 mg, or 300 mg, e.g., 25 mg to 100 mg, 200 mg, or 300 mg, e.g., 50 mg to 100 mg, 200 mg, or 300 mg, of (+)-1-(3,4-dichlorophenyl)-3-azabicyclo[3.1.0]hexane, in free or pharmaceutically acceptable salt form, substantially free of the corresponding (−) enantiomer.
2.12 Method 2 or 2.1-2.11 comprising administering 75 mg to 1000 mg, e.g., 100 mg to 600 mg, e.g., 100 mg to 400 mg, e.g., 100 mg to 200 mg, of (+)-1-(3,4-dichlorophenyl)-3-azabicyclo[3.1.0]hexane, in free or pharmaceutically acceptable salt form, substantially free of the corresponding (−) enantiomer.
2.13 Method 2 or 2.1-2.12 comprising administering 25 mg to 600 mg, 50 mg to 600 mg, e.g., 25 mg to 200 mg, e.g., 50 mg to 200 mg, e.g., 50 mg to 100 mg, e.g., 100 mg to 600 mg, e.g., 100 mg to 400 mg, e.g., 100 mg to 200 mg, of (+)-1-(3,4-dichlorophenyl)-3-azabicyclo[3.1.0]hexane, in free or pharmaceutically acceptable salt form, substantially free of the corresponding (−) enantiomer.
2.14 Method 2 or 2.1-2.13 comprising administering 100 mg to 600 mg, e.g., 100 mg to 400 mg, e.g., 100 mg to 300 mg, e.g., 100 mg to 200 mg, e.g., 100, 200, 300, or 400 mg, of (+)-1-(3,4-dichlorophenyl)-3-azabicyclo[3.1.0]hexane, in free or pharmaceutically acceptable salt form, substantially free of the corresponding (−) enantiomer.
2.15 Method 2 or 2.1-2.14 comprising administering 100 mg to 200 mg, e.g., 100 mg, of (+)-1-(3,4-dichlorophenyl)-3-azabicyclo[3.1.0]hexane, in free or pharmaceutically acceptable salt form, substantially free of the corresponding (−) enantiomer.
2.16 Method 2 or 2.1-2.15 comprising administering (+)-1-(3,4-dichlorophenyl)-3-azabicyclo[3.1.0]hexane, in free or pharmaceutically acceptable salt form, substantially free of the corresponding (−) enantiomer, once, twice, three, or four times daily.
2.17 Method 2 or 2.1-2.16 comprising administering (+)-1-(3,4-dichlorophenyl)-3-azabicyclo[3.1.0]hexane, in free or pharmaceutically acceptable salt form, substantially free of the corresponding (−) enantiomer, at least once daily.
2.18 Method 2 or 2.1-2.17 comprising administering (+)-1-(3,4-dichlorophenyl)-3-azabicyclo[3.1.0]hexane, in free or pharmaceutically acceptable salt form, substantially free of the corresponding (−) enantiomer, at least twice daily.
2.19 Method 2 or 2.1-2.18 comprising administering (+)-1-(3,4-dichlorophenyl)-3-azabicyclo[3.1.0]hexane, in free or pharmaceutically acceptable salt form, substantially free of the corresponding (−) enantiomer, at least three times daily.
2.20 Method 2 or 2.1-2.19 comprising administering (+)-1-(3,4-dichlorophenyl)-3-azabicyclo[3.1.0]hexane, in free or pharmaceutically acceptable salt form, substantially free of the corresponding (−) enantiomer, four times daily.
2.21 Method 2 or 2.1-2.20 comprising administering (+)-1-(3,4-dichlorophenyl)-3-azabicyclo[3.1.0]hexane, in free or pharmaceutically acceptable salt form, substantially free of the corresponding (−) enantiomer, 24 hours or less, e.g., 12 hours, e.g., 10 hours, e.g., 8 hours, e.g., 6 hours, e.g., 4 hours, e.g., 3 hours, e.g., 2 hours, e.g., 1 hour or less, before sexual activity.
2.22 Method 2 or 2.1-2.21 wherein (+)-1-(3,4-dichlorophenyl)-3-azabicyclo[3.1.0]hexane, in free or pharmaceutically acceptable salt form, substantially free of the corresponding (−) enantiomer, is administered concurrently or sequentially, in either order, with another treatment for female sexual dysfunction.
2.23 Method 2 or 2.1-2.22 wherein (+)-1-(3,4-dichlorophenyl)-3-azabicyclo[3.1.0]hexane, in free or pharmaceutically acceptable salt form, substantially free of the corresponding (−) enantiomer, is administered concurrently or sequentially, in either order, with a hormone or a derivative thereof (e.g., estrogen, testosterone (e.g., micronized oral testosterone), methyltestosterone, testosterone propionate, dehydroepiandrosterone, dehydroepiandrosterone sulfate), a prostaglandin (e.g., alprostadil), a melanocortin agonist (e.g., bremelanotide), a phosphodiesterase inhibitor (e.g., a PDE5 inhibitor, e.g., Viagra, or, e.g., a PDE1 inhibitor), a dopamine-norepinephrine reuptake inhibitor (e.g., bupropion), a 5-hydroxytryptamine₁ₐ receptor agonist, a dopamine agonist (e.g., apomorphine), a serotonin antagonist and reuptake inhibitor (e.g., trazodone), a lubricant, vitamin E, mineral oil, or a combination thereof
2.24 Method 2 or 2.1-2.23 wherein the female has major depressive disorder.
2.25 Method 2 or 2.1-2.24 wherein the female is taking an anti-hypertensive (e.g., a diuretic, a beta blocker, a calcium-channel blocker, an anti-adrenergic), an anticholinergic (e.g., propantheline, methantheline), a barbiturate, a benzodiazepine (e.g, diazepam, alprazolam), an anti-depressant (e.g., a selective serotonin re-uptake inhibitor, a serotonin-norepinephrine re-uptake inhibitor, a tricyclic antidepressant, a tetracyclic antidepressant, or a monoamine oxidase inhibitor, e.g., a selective serotonin re-uptake inhibitor, e.g., paroxetine), a chemotherapeutic (e.g., cyclophosphamide, an anti-estrogen), or an opiate (e.g., morphine, codeine, methadone), e.g., wherein the female is taking an anti-depressant (e.g., a selective serotonin re-uptake inhibitor, a serotonin-norepinephrine re-uptake inhibitor, a tricyclic antidepressant, a tetracyclic antidepressant, or a monoamine oxidase inhibitor, e.g., a selective serotonin re-uptake inhibitor, e.g., paroxetine).
2.26 Method 2 or 2.1-2.25 wherein the sexual dysfunction is a side effect of a drug, e.g., an anti-hypertensive (e.g., a diuretic, a beta blocker, a calcium-channel blocker, an anti-adrenergic), an anticholinergic (e.g., propantheline, methantheline), a barbiturate, a benzodiazepine (e.g, diazepam, alprazolam), an anti-depressant (e.g., a selective serotonin re-uptake inhibitor, a serotonin-norepinephrine re-uptake inhibitor, a tricyclic antidepressant, a tetracyclic antidepressant, or a monoamine oxidase inhibitor, e.g., selective serotonin re-uptake inhibitor, e.g., paroxetine), a chemotherapeutic (e.g., cyclophosphamide, an anti-estrogen), or an opiate (e.g., morphine, codeine, methadone), e.g., wherein the sexual dysfunction is a side effect of an anti-depressant (e.g., a selective serotonin re-uptake inhibitor, a serotonin-norepinephrine re-uptake inhibitor, a tricyclic antidepressant, a tetracyclic antidepressant, or a monoamine oxidase inhibitor, e.g., a selective serotonin re-uptake inhibitor, e.g., paroxetine).
2.27 Method 2.25 or 2.26 comprising administering a subthreshold amount of (+)-1-(3,4-dichlorophenyl)-3-azabicyclo[3.1.0]hexane, in free or pharmaceutically acceptable salt form, substantially free of the corresponding (−) enantiomer.
2.28 Method 2.27 wherein the subthreshold amount is the subthreshold amount of (+)-1-(3,4-dichlorophenyl)-3-azabicyclo[3.1.0]hexane, in free or pharmaceutically acceptable salt form, substantially free of the corresponding (−) enantiomer, for treatment of depression.
2.29 Method 2.27 wherein the subthreshold amount is the subthreshold amount of (+)-1-(3,4-dichlorophenyl)-3-azabicyclo[3.1.0]hexane, in free or pharmaceutically acceptable salt form, substantially free of the corresponding (−) enantiomer, for treatment of female sexual dysfunction.
2.30 Any of Method 2.25-2.29 comprising administering 10 mg to 400 mg, e.g., 25 mg to 400 mg, e.g., 25 mg to 300 mg, e.g., 50 mg to 400 mg, e.g., 50 mg to 300 mg, e.g., 50 mg to 200 mg, e.g., 50 to 100 mg, e.g., 100 mg to 400 mg, e.g., 100 mg to 300 mg, e.g., 100 mg to 200 mg, e.g., 100 mg, 200 mg, 300 mg, or 400 mg, of (+)-1-(3,4-dichlorophenyl)-3-azabicyclo[3.1.0]hexane, in free or pharmaceutically acceptable salt form, substantially free of the corresponding (−) enantiomer.

Also provided herein is use of (+)-1-(3,4-dichlorophenyl)-3-azabicyclo[3.1.0]hexane, in free or pharmaceutically acceptable salt form, substantially free of the corresponding (−) enantiomer, for treatment or prophylaxis of female sexual dysfunction, e.g., for use in any of Method 2 or 2.1-2.30.

Also provided herein is use of (+)-1-(3,4-dichlorophenyl)-3-azabicyclo[3.1.0]hexane, in free or pharmaceutically acceptable salt form, substantially free of the corresponding (−) enantiomer, in the manufacture of a medicament for treatment or prophylaxis of female sexual dysfunction, e.g., for use in any of Method 2 or 2.1-2.30.

Also provided is a pharmaceutical composition comprising (+)-1-(3,4-dichlorophenyl)-3-azabicyclo[3.1.0]hexane, in free or pharmaceutically acceptable salt form, substantially free of the corresponding (−) enantiomer, in combination with a pharmaceutically acceptable diluent or carrier for use in treatment or prophylaxis of female sexual dysfunction, e.g., for use in any of Method 2 or 2.1-2.30.

Also provided herein is use of (+)-1-(3,4-dichlorophenyl)-3-azabicyclo[3.1.0]hexane, in free or pharmaceutically acceptable salt form, substantially free of the corresponding (−) enantiomer, for treating female sexual dysfunction, e.g., for use in any of Method 2 or 2.1-2.30.

Also provided herein is use of (+)-1-(3,4-dichlorophenyl)-3-azabicyclo[3.1.0]hexane, in free or pharmaceutically acceptable salt form, substantially free of the corresponding (−) enantiomer, in the manufacture of a medicament for treating female sexual dysfunction, e.g., for use in any of Method 2 or 2.1-2.30.

Also provided is a pharmaceutical composition comprising (+)-1-(3,4-dichlorophenyl)-3-azabicyclo[3.1.0]hexane, in free or pharmaceutically acceptable salt form, substantially free of the corresponding (−) enantiomer, in combination with a pharmaceutically acceptable diluent or carrier for use in treating female sexual dysfunction, e.g., for use in any of Method 2 or 2.1-2.30.

A dose or method of administration of the dose of the present disclosure is not particularly limited. Dosages employed in practicing the present disclosure will of course vary depending, e.g. on the mode of administration and the therapy desired. In general, satisfactory results, e.g. for the treatment of female sexual dysfunction are indicated to be obtained on oral administration at dosages of the order from about 0.01 to 2.0 mg/kg. In larger females, for example human females, an indicated daily dosage for oral administration will accordingly be in the range of from about 0.75 mg to 150 mg, conveniently administered once, or in divided doses 2 to 4 times, daily or in sustained release form. Unit dosage forms for oral administration thus for example may comprise from about 0.2 mg to 75 mg or 150 mg, e.g. from about 0.2 mg or 2.0 mg or 50 mg or 75 mg or 100 mg to 200 mg or 500 mg of (+)-1-(3,4-dichlorophenyl)-3-azabicyclo[3.1.0]hexane, in free or pharmaceutically acceptable salt form, together with a pharmaceutically acceptable diluent or carrier therefor. Unit dosage forms for oral administration thus for example may comprise from about 0.2 mg to 75 mg or 150 mg, e.g. from about 0.2 mg or 2.0 mg or 50 mg or 75 mg or 100 mg to 200 mg or 500 mg of (+)-1-(3,4-dichlorophenyl)-3-azabicyclo[3.1.0]hexane, in free or pharmaceutically acceptable salt form, substantially free of the corresponding (−) enantiomer, together with a pharmaceutically acceptable diluent or carrier therefor.

(+)-1-(3,4-dichlorophenyl)-3-azabicyclo[3.1.0]hexane, in free or pharmaceutically acceptable salt form, may be administered by any suitable route, including orally, parenterally, transdermally, inhalation, slow release, controlled release, although various other known delivery routes, devices and methods can likewise be employed. (+)-1-(3,4-dichlorophenyl)-3-azabicyclo[3.1.0]hexane, in free or pharmaceutically acceptable salt form, substantially free of the corresponding (−) enantiomer, may be administered by any suitable route, including orally, parenterally, transdermally, inhalation, slow release, controlled release, although various other known delivery routes, devices and methods can likewise be employed. In some embodiments, provided is a sustained release pharmaceutical composition, e.g., an oral sustained release pharmaceutical composition, comprising (+)-1-(3,4-dichlorophenyl)-3-azabicyclo[3.1.0]hexane, in free or pharmaceutically acceptable salt form, which provides therapeutically effective levels of (+)-1-(3,4-dichlorophenyl)-3-azabicyclo[3.1.0]hexane over a sustained delivery period of approximately 6 hours or longer, e.g., 8 hours or longer, e.g., 12 hours or longer, e.g., 18 hours or longer, e.g., 24 hours or longer. In some embodiments, provided is a sustained release pharmaceutical composition, e.g., an oral sustained release pharmaceutical composition, comprising (+)-1-(3,4-dichlorophenyl)-3-azabicyclo[3.1.0]hexane, in free or pharmaceutically acceptable salt form, substantially free of the corresponding (−) enantiomer, which provides therapeutically effective levels of (+)-1-(3,4-dichlorophenyl)-3-azabicyclo[3.1.0]hexane over a sustained delivery period of approximately 6 hours or longer, e.g., 8 hours or longer, e.g., 12 hours or longer, e.g., 18 hours or longer, e.g., 24 hours or longer. In some embodiments, provided is an immediate release pharmaceutical composition, e.g., an oral immediate release pharmaceutical composition, comprising (+)-1-(3,4-dichlorophenyl)-3-azabicyclo[3.1.0]hexane, in free or pharmaceutically acceptable salt form. In some embodiments, provided is an immediate release pharmaceutical composition, e.g., an oral immediate release pharmaceutical composition, comprising (+)-1-(3,4-dichlorophenyl)-3-azabicyclo[3.1.0]hexane, in free or pharmaceutically acceptable salt form, substantially free of the corresponding (−) enantiomer.

Pharmaceutical compositions comprising (+)-1-(3,4-dichlorophenyl)-3-azabicyclo[3.1.0]hexane, in free or pharmaceutically acceptable salt form, may be prepared using conventional diluents or excipients and techniques known in the galenic art. Pharmaceutical compositions comprising (+)-1-(3,4-dichlorophenyl)-3-azabicyclo[3.1.0]hexane, in free or pharmaceutically acceptable salt form, substantially free of the corresponding (−) enantiomer, may be prepared using conventional diluents or excipients and techniques known in the galenic art. Thus, oral dosage forms may include tablets, capsules, solutions, suspensions, and the like.

Where two active agents are administered, the therapeutically effective amount of each agent may be below the amount needed for activity as monotherapy. Accordingly, a subthreshold amount (i.e., an amount below the level necessary for efficacy as monotherapy) may be considered therapeutically effective. Indeed, an advantage of administering different agents with different mechanisms of action and different side effect profiles may be to reduce the dosage and side effects of either or both agents, as well as to enhance or potentiate their activity as monotherapy.

Examples of sexual functioning questionnaires to detect sexual dysfunction are described in Labbate, L. et al., “Sexual Dysfunction in Male psychiatric Outpatients: Validity of the Massachusetts General Hospital Sexual Functioning Questionnaire,” Psychotherapy and Psychosomatics, 2001, 70, 221-225, which is incorporated by reference in its entirety.

### EXAMPLE

### Example 1

A multicenter, randomized, double blind, placebo- and paroxetine-controlled trial of amitifadine for major depressive disorder (MDD) non-responsive to an adequate trial of an antidepressant therapy in the current depressive episode. A sequential parallel comparison design (SPCD) is utilized. All patients are initially randomized to receive either amitifadine 50 mg or 100 mg, paroxetine 40 mg, or placebo. The study is 12 weeks in duration, with two 6-week phases. The primary outcome measure is change on the Montgomery Asberg Depression Rating Scale (MADRS). Secondary outcomes include Hamilton Rating Scale for Depression (HAM-D), Clinical Global Impression of Improvement (CGI-I), Sexual Functioning Inventory (SFI), Clinical Global Impression (Improvement and Severity versions), and the Massachusetts General Hospital (MGH) Cognitive and Physical Functioning Questionnaire (MGH-CPFQ). Male and female patients, between the ages of 18-65, are eligible who had MDD, based on the Structured Clinical Interview for DSM Disorders (SCID) interview, and validated by remote raters, a history of non-response to an adequate trial of an antidepressant during the current episode of MDD (an adequate dose for >8 weeks), and a HAM-D-17 score of >18 at screening.

In addition to standard inclusion criteria the trial requires: 1) diagnosis of MDD, based on the Structured Clinical Interview for DSM Disorders Clinical Trials version (SCID-CT), validated by remote independent raters using the SAFER criteria interview, 2) one and only one antidepressant therapy (ADT) failure in the current episode of MDD, treated with an adequate dose of one of an allowed standard antidepressant (paroxetine or an monoamine oxidase inhibitor (MAOI) not allowed) during the current episode for >6 weeks, with the same, adequate dose for >4 weeks, 3) HAM-D17≧18 at the end of screening phase, 4)<25% reduction in Quick Inventory of Depressive Symptoms Self-Rated (QIDS-SR) score between the screen and baseline visits. Inadequate response: defined as <50% reduction in depressive symptom severity, assessed by the MGH Antidepressant Treatment Response Questionnaire (ATRQ) administered by remote, independent raters (Targum, S. et al., CNS Neuroscience & Therapeutics, 2010, 16 (5), 322-325). An adequate trial is defined as an ADT>6 weeks at least at the minimum dose specified in the MGH ATRQ. Patients do not have to be on the failed ADT medication at the time of screening visit.

Additional criteria for defining non-response for patients in Phase 2: Among patients randomized to receive placebo in Phase 1 and to be re-randomized to receive placebo, amitifadine 50 mg/day or amitifadine 100 mg/day in Phase 2, only those meeting non-response criteria are added to the primary efficacy sample. Placebo non-responders are defined as those patients who fail to achieve at least a 50% decrease from Phase 1 baseline in their MADRS score at visit 8 (Week 6), and had a MADRS score of ≧16 at visit 8 (Week 6) of Phase 1. The SCID-CT is administered by study clinicians at the screening visit to diagnose Axis I disorders. The remote, independent raters also administer the SAFER Criteria Inventory (SCI) during the screening phase to assess whether the MDD is a “valid” clinical entity.

Efficacy and Safety Assessments

The primary efficacy assessment is the MADRS, administered at every study visit and clinicians complete it by using a structured interview guide developed by Massachusetts General Hospital (MGH). The primary tolerability assessment is the change in the SFI, completed by the patient at every study visit. The MGH-CPFQ, a brief self-report inventory to assess rates of significant cognitive symptoms, sleepiness and fatigue, is filled out by patients at every visit. The Columbia-Suicide Severity Rating Scale (C-SSRS) (Posner K. et al., American Journal of Psychiatry, 2011, 168 (12)1266-77) is administered at every visit. Vital signs are recorded at each visit; physical exam is performed at screen and week 12 (or early termination). Laboratory tests are obtained at screen, baseline, and weeks 2, 4, 6, 8, 10, 12 (or early termination). Twelve-lead ECGs are obtained at screen, baseline, and weeks 3, 6, 9, 12 (or early termination). Consumptive habits (smoking, alcohol, and caffeinated beverages) are recorded at every visit. Adverse events and concomitant medications are collected at every visit.

Results:

A total of 342 patients are randomized. In the primary analysis, there is no significant difference in MADRS change from baseline for amitifadine 50 mg or 100 mg compared to placebo. There is a significant difference between paroxetine and placebo, verifying the internal validity of the trial. There are no significant differences on the CGI-Improvement between amitifadine 50 mg or 100 mg and placebo; there is a significant difference on the CGI-I between paroxetine and placebo at both 6 and 12 weeks.

Despite lack of efficacy on the primary outcome, exploratory analyses does suggest potential signals for a dose related antidepressant effect of amitifadine. In exploratory analyses, paroxetine demonstrates significantly better improvement compared to both placebo and amitifadine 50 mg, although the difference between paroxetine and amitifadine 100 mg is non-significant.

However, the trial is not powered as a non-inferiority trial. For patients with confirmed adherence based on PK levels, the improvement in terms of change on the MADRS from baseline with amitifadine 100 mg is similar to that seen with paroxetine. Neither dose is associated with significant changes in heart rate or blood pressure.

Those receiving amitifadine 100 mg had significantly less overall worsening of sexual function than those receiving paroxetine. On most individual items of the SFI, amitifadine 100 mg is significantly less impairing than paroxetine, such as anorgasmia, arousal, and overall satisfaction. In post-hoc analyses of the SFI by gender, women experience significant improvement on 100 mg amitifadine from baseline compared with placebo. See Tables 1 and 2.

TABLE 1
LOCF Analysis of Mean Change from Baseline SFI

| | Compare vs. | | |
|---|---|---|---|
| SFI Item | Paroxetine | Both Genders | p-Values |
| 1 | Amitifadine 100 | −1.02 | 0.1054 |
| | Paroxetine | −0.57 | |
| 2 | Amitifadine 100 | −1.00 | 0.0326 |
| | Paroxetine | −0.45 | |
| 3 | Amitifadine 100 | −0.80 | 0.0391 |
| | Paroxetine | −0.27 | |
| 4 | Amitifadine 100 | −0.79 | 0.0621 |
| | Paroxetine | −0.13 | |
| 5 | Amitifadine 100 | −1.00 | 0.0032 |
| | Paroxetine | −0.23 | |
| SFI TOTAL | Amitifadine 100 | −3.86 | 0.0134 |
| | Paroxetine | −1.52 | |

TABLE 2
LOCF Analysis of Mean Change from Baseline SFI

| | Compare vs. | | |
|---|---|---|---|
| SFI Item | Placebo | Female | Male |
| 1 | Amitifadine 100 | −1.16 | −0.83 |
| | Placebo | −0.45 | −0.92 |
| | p-Value | 0.0147 | 0.1018 |
| 2 | Amitifadine 100 | −1.12 | −0.83 |
| | Placebo | −0.27 | −0.83 |
| | p-Value | 0.0276 | 0.9847 |
| 3 | Amitifadine 100 | −0.97 | −0.58 |
| | Placebo | −0.19 | −0.85 |
| | p-Value | 0.0042 | 0.4317 |
| 4 | Amitifadine 100 | | −0.79 |
| | Placebo | | −0.56 |
| | p-Value | | 0.4321 |
| 5 | Amitifadine 100 | −1.09 | −0.88 |
| | Placebo | −0.41 | −0.69 |
| | p-Value | 0.0131 | 0.5906 |
| SFI Total | Amitifadine 100 | −4.41 | −3.12 |
| | Placebo | −1.25 | −3.29 |
| | p-Value | 0.0016 | 0.8936 |

1. A method of prophylaxis or treatment of female sexual dysfunction in a female in need thereof comprising administering a therapeutically effective amount of (+)-1-(3,4-dichlorophenyl)-3-azabicyclo[3.1.0]hexane, in free or pharmaceutically acceptable salt form, to the female.

2-6. (canceled)

7. The method of claim 1, wherein (+)-1-(3,4-dichlorophenyl)-3-azabicyclo[3.1.0]hexane in pharmaceutically acceptable salt form is an acid addition salt.

8. The method of claim 7, wherein (+)-1-(3,4-dichlorophenyl)-3-azabicyclo[3.1.0]hexane in pharmaceutically acceptable salt form is (+)-1-(3,4-dichlorophenyl)-3-azabicyclo[3.1.0]hexane hydrochloride.

9. The method of claim 1, wherein (+)-1-(3,4-dichlorophenyl)-3-azabicyclo[3.1.0]hexane in pharmaceutically acceptable salt form is crystalline.

10. The method of claim 8, wherein (+)-1-(3,4-dichlorophenyl)-3-azabicyclo[3.1.0]hexane hydrochloride is Polymorph A.

11-14. (canceled)

15. The method of claim 1 comprising administering 25 mg to 600 mg of (+)-1-(3,4-dichlorophenyl)-3-azabicyclo[3.1.0]hexane, in free or pharmaceutically acceptable salt form.

16. The method of claim 15 comprising administering 100 mg to 600 mg of (+)-1-(3,4-dichlorophenyl)-3-azabicyclo[3.1.0]hexane, in free or pharmaceutically acceptable salt form.

17. The method of claim 16 comprising administering 100 mg to 200 mg of (+)-1-(3,4-dichlorophenyl)-3-azabicyclo[3.1.0]hexane, in free or pharmaceutically acceptable salt form.

18-22. (canceled)

23. The method of claim 1 comprising administering (+)-1-(3,4-dichlorophenyl)-3-azabicyclo[3.1.0]hexane, in free or pharmaceutically acceptable salt form, 24 hours or less before sexual activity.

24. The method of claim 1, wherein (+)-1-(3,4-dichlorophenyl)-3-azabicyclo[3.1.0]hexane, in free or pharmaceutically acceptable salt form, is administered concurrently or sequentially, in either order, with another treatment for female sexual dysfunction.

25. The method of claim 1, wherein (+)-1-(3,4-dichlorophenyl)-3-azabicyclo[3.1.0]hexane, in free or pharmaceutically acceptable salt form, is administered concurrently or sequentially, in either order, with a hormone or a derivative thereof, a prostaglandin, a melanocortin agonist, a phosphodiesterase inhibitor, a dopamine-norepinephrine reuptake inhibitor, a 5-hydroxytryptamine₁ₐ receptor agonist, a dopamine agonist, a serotonin antagonist and reuptake inhibitor, a lubricant, vitamin E, mineral oil, or a combination thereof.

26. The method of claim 1, wherein the female has major depressive disorder.

27. The method of claim 1, wherein the female is taking an anti-hypertensive, an anticholinergic, a barbiturate, a benzodiazepine, an anti-depressant, a chemotherapeutic, or an opiate.

28. The method of claim 27, wherein the female is taking an anti-depressant.

29. The method of claim 1, wherein the sexual dysfunction is a side effect of a drug.

30. The method of claim 1, wherein the sexual dysfunction is a side effect of an anti-depressant.

31. The method of claim 1 comprising administering a subthreshold amount of (+)-1-(3,4-dichlorophenyl)-3-azabicyclo[3.1.0]hexane, in free or pharmaceutically acceptable salt form, wherein the subthreshold amount is a subthreshold amount of (+)-1-(3,4-dichlorophenyl)-3-azabicyclo[3.1.0]hexane, in free or pharmaceutically acceptable salt form, for treatment of depression.

32-46. (canceled)
